(19) United States
(12) Patent Application Publication
(10) Pub. No.: US 2015/0173685 A1
(43) Pub. Date: Jun. 25, 2015

(54) TRIAGE SCORING SYSTEM

(71) Applicant: THE BINDING SITE GROUP LIMITED, Birmingham, West Midlands (GB)

(72) Inventors: Richard Hughes, Birmingham (GB); Stephen Harding, Birmingham (GB)

(21) Appl. No.: 14/415,884

(22) PCT Filed: Jul. 10, 2013
(86) PCT No.: PCT/GB2013/051828

§ 371 Date: Jan. 20, 2015

(30) Foreign Application Priority Data
Jul. 20, 2012 (GB) 1212900.3

(51) Int. Cl. A61B 5/00; G01N 33/68; A61B 5/16; A61B 5/20; A61B 5/0205; A61B 5/145

(52) U.S. Cl. CPC A61B 5/7275; A61B 5/02055; A61B 5/14542; A61B 5/4824; A61B 5/165; A61B 5/208; G01N 33/6893; G01N 33/6857; A61B 5/021

(57) ABSTRACT

The application discloses a method of determining the severity of symptoms in a patient comprising (i) producing a triage score, such as an early warning score (EWS) modified early warning score (MEWS), paediatric early warning score (PEWS), NHS early warning score (NEWS), simple clinical score (SCS), rapid emergency score (REMS) or mortality in emergency department sepsis score for the patient, (ii) measuring an amount of free light chains (FLC), preferably combined free light chains (cFLC), in a sample from the patient, and (iii) using the triage score and the amount of FLC measured to assess the severity of symptoms in the patient. This also allows patients to be triaged to provide better treatment of them.

The invention relates to a method of determining the severity of symptoms in a patients by producing a triage score, such as an early warning score (EWS) or modified early warning score (MEWS) for the patient and measuring an amount of free light chains (FLC), such as combined free light chains (cFLC) in a sample from the patient.

Antibodies comprise heavy chains and light chains. They usually have a two-fold symmetry and are composed of two identical heavy chains and two identical light chains, each containing variable and constant region domains. The variable domains of each light-chain/heavy-chain pair combine to form an antigen-binding site, so that both chains contribute to the antigen-binding specificity of the antibody molecule. Light chains are of two types, κ and λ, and any given antibody molecule is produced with either light chain but never both. There are approximately twice as many κ as λ, molecules produced in humans, but this is different in some mammals. Usually the light chains are attached to heavy chains. However, some unattached “free light chains” are detectable in the serum or urine of individuals. FLC may be specifically identified by raising antibodies against the surface of the free light chain that is normally hidden by the binding of the light chain to the heavy chain. In FLC this surface is exposed, allowing it to be detected immunologically. Commercially available kits for the detection of κ or λ, FLC include, for example, “Freelite™”, manufactured by The Binding Site Group Limited, Birmingham, United Kingdom. The Applicants have previously identified that measuring the amount of free κ, free λ, and/or free κ/free λ, ratios, allows the detection of monoclonal gammopathies in patients. It has been used, for example, as an aid in the diagnosis of intact immunoglobulin multiple myeloma (MM), light chain MM, non-secretory MM, AL amyloidosis, light chain deposition disease, smouldering MM, plasmacytoma and MGUS (monoclonal gammopathies of undetermined significance). Detection of FLC has also been used, for example, as an aid to the diagnosis of other B-cell dyscrasia and indeed as an alternative to urinary Bence Jones protein analysis for the diagnosis of monoclonal gammopathies in general.

Conventionally, an increase in one of the λ, or κ light chains is looked for. For example, multiple myelomas result from the monoclonal multiplication of a malignant plasma cell, resulting in an increase in a single type of cell producing a single type of immunoglobulin. This results in an increase in the amount of FLC, either λ, or κ, observed within an individual. This increase in concentration may be determined, and usually the ratio of the free κ to free λ, is determined and compared with the normal range. This aids in the diagnosis of monoclonal disease. Moreover, the FLC assays may also be used for the following of treatment of the disease in patients. Prognosis of, for example, patients after treatment for AL amyloidosis may be carried out.

Katzmann et at (Clin. Chem. (2002); 48(9): 1437-1944) discuss serum reference intervals and diagnostic ranges for free κ and free λ, immunoglobulins in the diagnosis of monoclonal gammopathies. Individuals from 21-90 years of age were studied by immunoassay and compared to results obtained by immunofixation to optimise the immunoassay for the detection of monoclonal FLC in individuals with B-cell dyscrasia.

The amount of κ and λ, FLC and the κ/λ, ratios were recorded allowing a reference interval to be determined for the detection of B-cell dyscrasias.

The Applicants had identified that assaying for FLC and especially total FLC can be used to predict long-term survival of individuals over a period of a number of years, even when the individual is an apparently healthy subject. They have found that FLC concentration is statistically, significantly linked to long-term survival. Moreover, this link appears to be similar or better than the link for existing long-term survival prognostic markers such as cholesterol, creatinine, cystatin C and C-reactive protein.

More recently cFLC have been shown to be prognostic of a number of clinical scenarios, including chronic kidney disease (Stringer S. Haematology Reports (2010) 2(S2) page 6). Elevated cFLC in samples of serum in patients referred to a haematology unit have been shown to correlate to increased frequency of death in patients after 100 days (Basu S et at Haematologica (2011) 96 (S2):0805a).

Triage scoring systems, such as EWS and MEWS are simple guides used by hospital nursing and medical staff as well as emergency medical services to determine the degree of illness of a patients. Typically a systolic blood pressure (BP), heart rate (beats per minute—BPM), respiratory rate (Respiration Per Minute) and body temperature (° C.) are scored, optionally together with an observation of the level of consciousness. These are compared to predetermined normal levels to produce a numerical score.

The table below shows a typical MEWS scoring system:

TABLE 1

| | Score | | | | | | |
|---|---|---|---|---|---|---|---|
| | 3 | 2 | 1 | 0 | 1 | 2 | 3 |
| Systolic BP | <45% | 30% | 15% down | Normal for patients | 15% up | 30% | >45% |
| Heart rate | — | <40 | 41-50 | 51-100 | 101-110 | 111-129 | >130 |
| Respiratory | — | <9 | — | 9-14 | 15-20 | 21-29 | >30 |
| rate (RPM) | | | | | | | |
| Temperature | — | <35 | — | 35.0-38.4 | — | >38.5 | — |
| (° C.) | | | | | | | |
| AVPU* | — | — | — | A | V | P | U |

*level of consciousness A = Alert, V = Voice, P = Pain, U = Unconscious

A score of 4 or more in the above system indicates an increased risk of death or admission to an intensive care unit for increased medical intervention. See for example, Subbe C P et at (QJM (2001) 94, 521-526).

The Applicant noticed that there are problems to these scoring systems. Patients with a low MEWS score have occasionally been shown to require hospital admissions or be retained in hospital for further treatment (Birch V C et at Emerg. Med. J. (2008) 25 (10) 674-8.

If patients are not scored properly then they may not be treated correctly.

Moreover, in some countries, such as England, hospitals are fined if patients are discharged and then have to be readmitted. Hence the use of an accurate EWS or MEWS system to identify patients needing additional treatment and to reduce readmission rates is required.

The Applicant realised that using a EWS or MEWS system with FLC levels could be used to improve the ability to assess the severity of an illness in a patient.

The invention provides a method of determining the severity of symptoms in a patient comprising (i) producing a triage score, (ii) measuring an amount of FLC, preferably combined free light chains (cFLC), in a sample from the patient, and (III) using the EWS or MEWS score and the amount of FLC measured to assess the severity of symptoms in the patient.

The triage score may, for example, be an early warning score (EWS), modified early warning score (MEWS), paediatric early warning score (PEWS), NHS early warning score (NEWS), simple clinical score (SCS), rapid emergency score (REMS) or mortality in emergency department sepsis score, all of which are generally known in the art.

In PEWS for example, respiratory, cardiovascular and behavior (playing, sleeping or irritable) are used as markers for the score. The SCS system looks at factors including age, oxygen saturation, blood pressure, fever, ECG abnormalities and other factors such as mental status, stroke and ability to stand.

One, two, three or all four of systolic blood pressure, heart rate, respiratory rate, and/or body temperature may be measured to produce the score. An observation, such as the level of consciousness of the patient may be assessed and scored.

One or more of blood oxygen saturation, ECG, urine output and/or a pain score may also be used to produce the triage score.

A typical scoring system is shown on the table above. The blood pressure, heart rate, respiratory rate and body temperature and other features tested may be measured using methods well known in the art. Typically they are non-invasive.

The patient may be an admission, such as an emergency admission, to a medical admissions unit, for example at a hospital.

The patient may also be a patient at a hospital awaiting discharge after treatment. In this case the invention is used as an assessment of whether further treatment, for example for undiagnosed conditions, should be carried out, rather than discharging the patient.

Measuring the severity of symptoms preferably means obtaining an indication of the likelihood of a the patient's symptoms causing illness, especially serious illness, or death within the short term, for example within 150, 100, 75, 50, 25 or fewer days from the date of assessment.

Accordingly, the method provides the further step of carrying out treatment or further diagnostic procedures of the patients where it is required, keeping the patient under medical observation for a further period of time or discharging the patient from medical supervision.

The amount of FLC may be compared to a predetermined normal range of FLC to indicate whether the amount of FLC is higher or lower than the normal range. This may be scored to produce a numerical score for the concentration, in a similar manner to the triage score, such as EWS or MEWS.

The FLC may be kappa or lambda FLC. However, preferably the total FLC concentration is measured as detecting kappa FLC or lambda FLC alone may miss, for example abnormally high levels of one or other FLC produced for example monoclonally in the patient.

Combined FLC means the total amount of free kappa plus free lambda light chains in a sample.

The term “total free light chains” means the amount of κ and λ, free light chains in the sample from the subject.

The sample is typically a sample of serum from the subject. However, whole blood, plasma, urine or other samples of tissue or fluids may also potentially be utilised.

Typically the FLC, such as total FLC, is determined by immunoassay, such as ELISA assays or utilising fluorescently labelled beads, such as Luminex™ beads. Alternatively, it may be used in the form of a lateral flow point of care test kit generally known in the art.

ELISA, for example uses antibodies to detect specific antigens. One or more of the antibodies used in the assay may be labelled with an enzyme capable of converting a substrate into a detectable analyte. Such enzymes include horseradish peroxidase, alkaline phosphatase and other enzymes known in the art. Alternatively, other detectable tags or labels may be used instead of, or together with, the enzymes. These include radioisotopes, a wide range of coloured and fluorescent labels known in the art, including fluorescein, Alexa fluor, Oregon Green, BODIPY, rhodamine red, Cascade Blue, Marina Blue, Pacific Blue, Cascade Yellow, gold; and conjugates such as biotin (available from, for example, Invitrogen Ltd, United Kingdom). Dye sols, metallic sols, chemiluminescent labels or coloured latex may also be used. One or more of these labels may be used in the ELISA assays according to the various inventions described herein, or alternatively in the other assays, labelled antibodies or kits described herein.

The construction of ELISA-type assays is itself well known in the art. For example, a “binding antibody” specific for the FLC is immobilised on a substrate. The “binding antibody” may be immobilised onto the substrate by methods which are well known in the art. FLC in the sample are bound by the “binding antibody” which binds the FLC to the substrate via the “binding antibody”.

Unbound immunoglobulins may be washed away.

In ELISA assays the presence of bound immunoglobulins may be determined by using a labelled “detecting antibody” specific to a different part of the FLC of interest than the binding antibody.

Flow cytometry may be used to detect the binding of the FLC of interest. This technique is well known in the art for, e.g. cell sorting. However, it can also be used to detect labelled particles, such as beads, and to measure their size. Numerous text books describe flow cytometry, such as Practical Flow Cytometry, 3rd Ed. (1994), H. Shapiro, Alan R. Liss, New York, and Flow Cytometry, First Principles (2nd Ed.) 2001, A. L. Given, Wiley Liss.

One of the binding antibodies, such as the antibody specific for FLC, is bound to a bead, such as a polystyrene or latex bead. The beads are mixed with the sample and the second detecting antibody. The detecting antibody is preferably labelled with a detectable label, which binds the FLC to be detected in the sample. This results in a labelled bead when the FLC to be assayed is present.

Other antibodies specific for other analytes described herein may also be used to allow the detection of those analytes.

Labelled beads may then be detected via flow cytometry. Different labels, such as different fluorescent labels may be used for, for example, the anti-free λ and anti-free κ antibodies. Other antibodies specific for other analytes described herein may also be used in this or other assays described herein to allow the detection of those analytes. This allows the amount of each type of FLC bound to be determined simultaneously or the presence of other analytes to be determined.

Alternatively, or additionally, different sized beads may be used for different antibodies, for example for different marker specific antibodies. Flow cytometry can distinguish between different sized beads and hence can rapidly determine the amount of each FLC or other analyte in a sample.

An alternative method uses the antibodies bound to, for example, fluorescently labelled beads such as commercially available Luminex™ beads. Different beads are used with different antibodies. Different beads are labelled with different fluorophore mixtures, thus allowing different analytes to be determined by the fluorescent wavelength. Luminex beads are available from Luminex Corporation, Austin, Tex., United States of America.

Preferably the assay used is a nephelometric or turbidimetric method. Nephelometric and turbidimetric assays for the detection of λ- or κ-FLC are generally known in the art. They have the best level of sensitivity for the assay. λ, and κ FLC concentrations may be separately determined or a single assay for total FLC arrived at. Such an assay contains anti-κ and anti-λ FLC antibodies typically at a 50:50 ratio.

Antibodies may also be raised against a mixture of free λ, and free κ light chains.

The amount of total FLC may be compared to a standard, predetermined value to determine whether the total amount is higher or lower than a normal value.

Preferably the method comprises detecting the amount of total FLC in the sample utilising an immunoassay, for example, by utilising a mixture of anti-free κ light chain and anti-free λ light chain antibodies or fragments thereof. Such antibodies may be in a ratio of 50:50 anti-κ: anti-λ antibodies. Antibodies, or fragments, bound to FLC may be detected directly by using labelled antibodies or fragments, or indirectly using labelled antibodies against the anti-free λ or anti-free κ antibodies.

The antibodies may be polyclonal or monoclonal. Polyclonal may be used because they allow for some variability between light chains of the same type to be detected as they are raised against different parts of the same chain. The production of polyclonal antibodies is described, for example in WO97/17372.

A level above 50 mg/L, especially more than 65 mg/ml is considered to show that the subject has an increased likelihood of overall death.

One or more additional markers may also be tested in the sample. These include albumin. The use of such assays in general is known in the art. The use of an additional marker is expected to provide further data and improve the accuracy of the prognosis or aid in the diagnosis of an underlying disease/medical problem. A concentration of albumin below 40 g/L, especially below 33 mg/L indicated an increased risk of death within 100 days without further treatment. Other markers include C-reactive protein (CRP) estimated glomerular filtration rate (eGFR), and erythrocyte sedimentation rate (ESR).

Fragment of antibodies, such as (Fab)₂ or Fab antibodies, which are capable of binding FLC may also be used.

The antibodies or fragments may be labelled, for example with a label as described above. Labelled anti-immunoglobulin binding antibodies or fragments thereof may be provided to detect anti-free λ or anti-free κ bound to FLC.

Kits may form part of a larger test assay kit comprising components for testing other markers, such as albumin etc., as described above. Antibodies for such markers may be provided.

The kit may comprise calibrator fluids to allow the assay to be calibrated at the ranges indicated. The calibrator fluids preferably contain predetermined concentrations of FLC, for example 6.25-200 mg/L. The kit may also be adapted by optimising the amount of antibody and “blocking” protein coated onto latex particles and, for example, by optimising concentrations of supplementary reagents such as polyethylene glycol (PEG) concentrations.

The kit may comprise, for example, a plurality of standard controls for the FLC or indeed other compounds such as albumin, which may be assayed. The standard controls may be used to validate a standard curve for the concentrations of the FLC or other components to be produced. Such standard controls confirm that the previously calibrated standard curves are valid for the reagents and conditions being used. They are typically used at substantially the same time as the assays of samples from subjects.

The assay kit may be a nephelometric or turbidimetric kit. It may be an ELISA, flow cytometry, fluorescent, chemiluminescent or bead-type assay or dipstick. Such assays are generally known in the art.

The invention will now be described by way of example only with reference to the following figures:

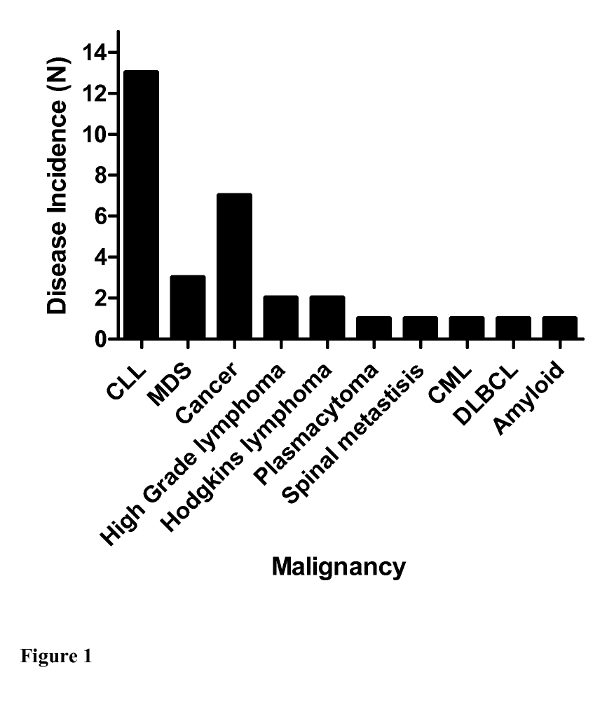

FIG. 1: Figure detailing the malignancies recorded for the study population.

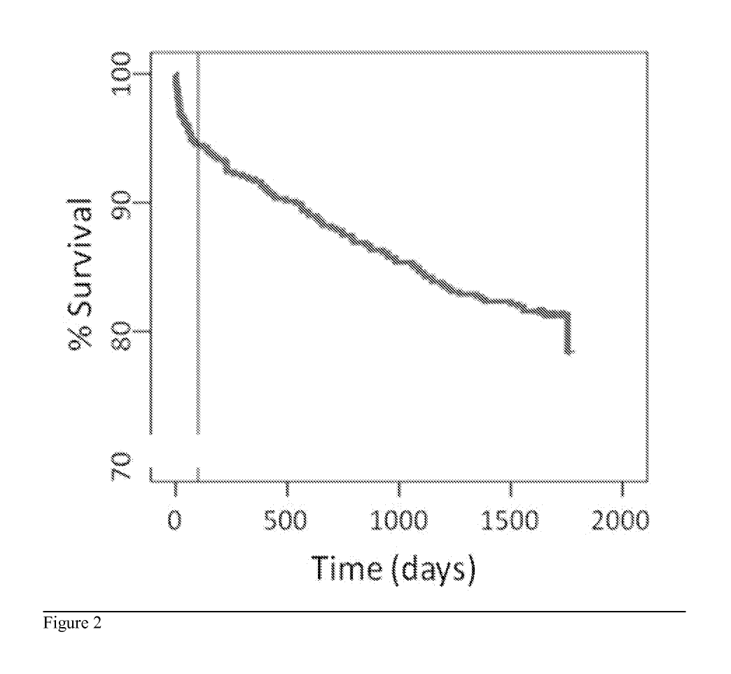

FIG. 2: Kaplan-Meier survival curve for all patients over the full period of follow up. The large number of deaths within the first 100 days is apparent. The vertical line indicates the 100 day time point.

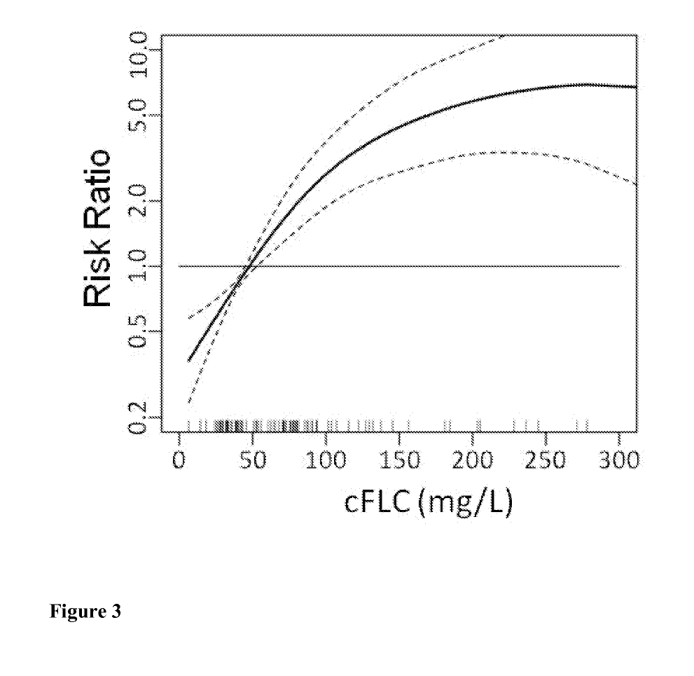

FIG. 3: Figure illustrating how the risk of death (within the full period of follow-up) varied with cFLC concentration (solid line). The broken lines represent the 95% confidence intervals.

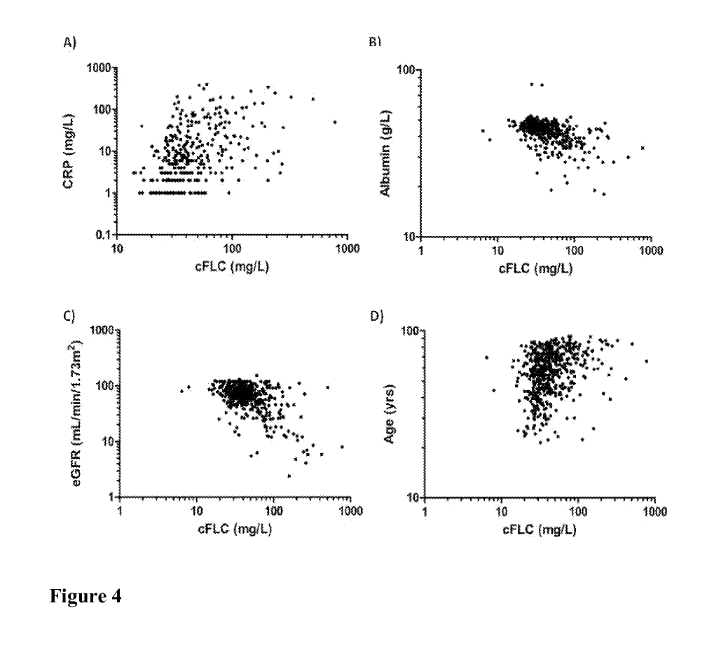

FIG. 4: Correlation analysis between cFLC and A) CRP r=0.44, p<0.001, B) Albumin r=−0.48, p<0.001, C) eGFR r=−0.38, p<0.001 and D) Age r=0.32, p<0.001.

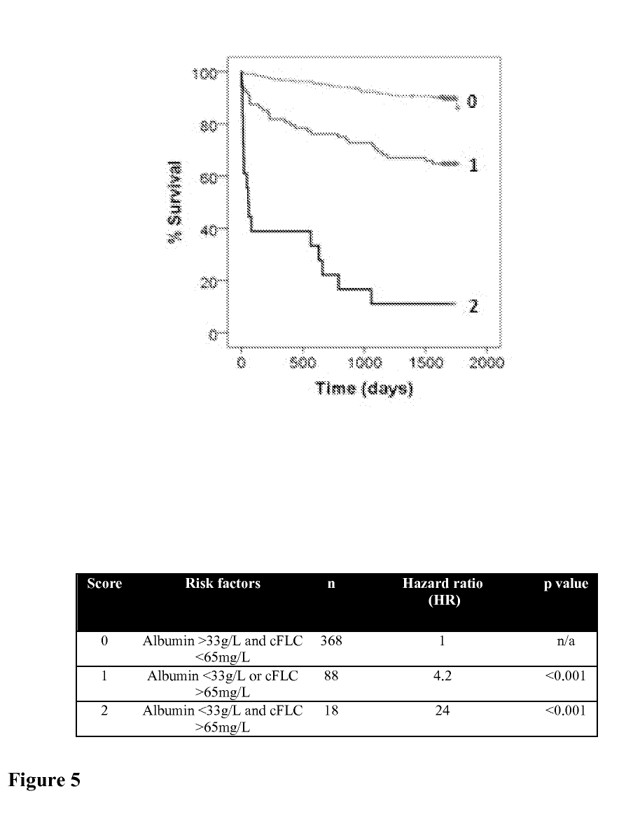

FIG. 5: A simple risk stratification model (Combylite-Risk Score) using low albumin (<33 g/L) and high cFLC concentrations (>65 mg/L) as risk factors. Probability of survival throughout the period of follow-up is compared for patients with 0 (dotted line), 1 (grey line) or 2 (black line) risk factors.

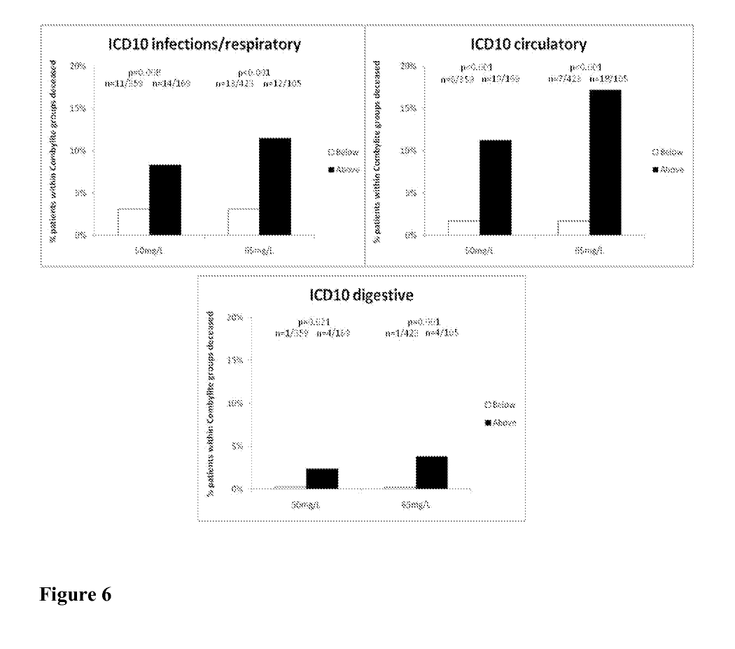

FIG. 6: Histograms illustrating the greater proportion of deaths recorded amongst patients with higher cFLC concentrations (>50 mg/L or >65 mg/L). This reached significance for the ICD10 death certificate classifications of infections/respiratory, circulatory and digestive.

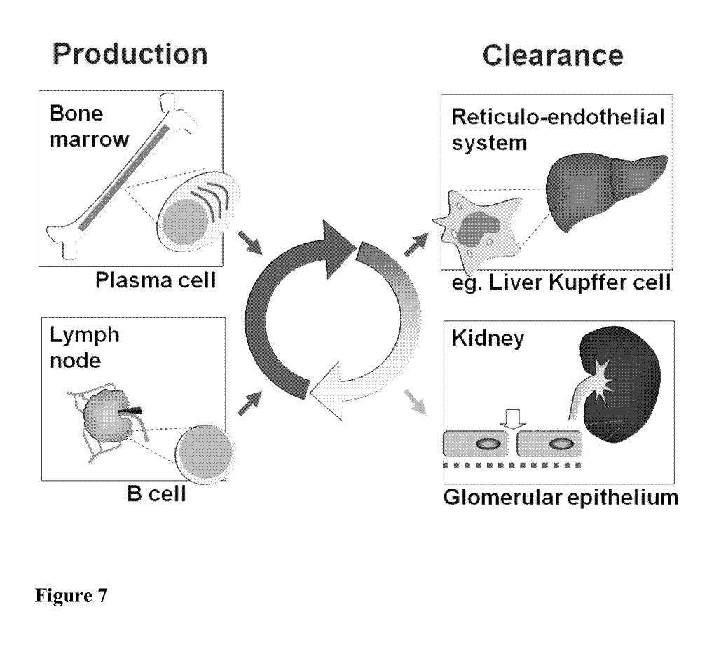

FIG. 7: Schematic illustrating the principal processes controlling the concentration of cFLC in the blood: production by plasma cells and earlier B-cells and clearance via the kidney and the reticulo-endothelial system. Pathologies which influence one or more of these processes could result in a change in the cFLC concentration.

### FLC CONCENTRATION IN SERUM IS ASSOCIATED WITH INCREASED MORTALITY

### Materials and Methods

The study was approved by the local Research Ethics Committee and the Research and Development Department of Royal Wolverhampton Hospitals, NHS Trust, UK.

### Study Population

Between Nov. 8, 2005 and Jan. 10, 2006 the laboratory received 723 sera with a request for SPE. Samples from paediatric patients, patients on immunoglobulin replacement, second and subsequent samples from the same patient, were excluded from the analysis. Also excluded were patients with evidence of a monoclonal gammopathy as indicated by an abnormal FLC ratio (<0.26 or >1.65; [Katzmann, J A, Clark, R J, Abraham, R S et al. Serum reference intervals and diagnostic ranges for free kappa and free lambda immunoglobulin light chains: relative sensitivity for detection of monoclonal light chains. Clin Chem 2002; 48; 1437-1444.]) or by detection of a monoclonal protein by SPE, confirmed by immunofixation electrophoresis (IFE). Therefore, the study comprised 527 selected patients.

### Laboratory Analyses

Sera were analysed for serum protein abnormalities by SPE (Sebia, UK). Serum IFE (Sebia) was performed on all samples with the presence of an abnormal SPE band or those with a high index of suspicion (unexplained hypogammaglobulinaemia, broad beta region, or low immunoglobulins supported by clinical observation). As part of an evaluation of FLC analysis in a diagnostic setting, FLC measurements (Freelite™, The Binding Site Group Ltd, Birmingham, UK) were made using a Siemens Dade-Behring Prospec nephelometer, in accordance with the manufacturer's instructions.

Total immunoglobulins (IgG, IgA, IgM) were measured by nephelometry (Dade-Behring). Normal range values used for the immunoglobulin concentrations were: IgG 6-16 g/L, IgA 0.8-4.0 g/L and IgM 0.5-2.0 g/L [Milford Ward, A., Sheldon, J., Rowbottom, A., and Wild, G. D. PRU Handbook of Clinical Immunochemistry. 9 ed. PRU Publications; 2007.]. Serum creatinine was determined for the majority of patients (497/527) (Roche; Modular). Estimated glomerular filtration rate (eGFR) was calculated using the MDRD equation [Levey, A S, Bosch, J P, Lewis, J B et al. A more accurate method to estimate glomerular filtration rate from serum creatinine: a new prediction equation. Modification of Diet in Renal Disease Study Group. Ann Intern Med 1999; 130; 461-470.]. C-reactive protein (CRP) was measured in 348/527 patients (Roche; Modular). Erythrocyte sedimentation rate (ESR) was determined in 390/527 patients (Starstedt; S-Sedivette®).

### Patient Follow-Up

In July 2010, 4 years and 6 months after the end of the original study, patient records were reviewed. For all patients, the date of the last follow-up or date of death was recorded and death certificates were obtained.

### Clinical Outcomes and Survival Analysis

Kaplan-Meier survival curves were constructed to identify factors influencing mortality over the period of follow-up.

Pearson correlation analysis was performed to determine the degree of correlation between the different biomarkers. Where available, established reference ranges were used, for inclusion of the different biomarkers into a Cox multivariate regression analysis as categorical variables (Tables 2a and 2b). For eGFR, a cut-off of <30 mL/min/1.73 m² (equivalent to CKD stages 4 and 5;[20]) was used. There is no published reference range for cFLC so a cut-off of 50 mg/L was selected, which approximates to the summation of the individual reference ranges for FLCκ and FLCλ (97.5 percentiles [17]). Additionally, Receiver-Operator-Characteristic (ROC) analysis was used to select a prognostically optimised cut-off. For age, a cut-off of 75 years was selected to leave 20% of patients in the higher risk group, comparable to the proportion outside the reference range for the other markers. Cox models were constructed for all deaths and for deaths within the first 100 days. A risk stratification model was constructed, for predicting the likelihood of deaths within 100 days, comprised of the two most significant, independent risk factors.

TABLE 2a
Univariate analysis: *factors indicated to be significant (p < 0.05)
predictors of mortality by univariate analysis.

| | All deaths | Deaths within 100 days |
|---|---|---|
| | p value | p value |
| Abnormal IgG | 0.323 | 0.647 |
| Abnormal IgA | 0.118 | 0.251 |
| Abnormal IgM | 0.666 | 0.164 |
| Abnormal IgGAM | 0.269 | 0.181 |
| Albumin <33 g/L | <0.001* | <0.001* |
| CRP >10 mg/L | 0.003* | <0.001* |
| ESR >12 | <0.001* | 0.034* |
| eGFR<30 mL/min/1.73 m² | <0.001* | <0.001* |
| Age >75 years | <0.001* | 0.03* |
| Gender (male) | 0.495 | 0.04* |
| cFLC >65 mg/L | <0.001* | <0.001* |

TABLE 2b
All factors found to be independent predictors using multivariate analysis.

| | | | Deaths | |
|---|---|---|---|---|
| | All deaths | | within 100 days | |
| | | Hazard | | Hazard |
| | p value | ratio (HR) | p value | ratio (HR) |
| eGFR <30 mL/min/1.73 m² | 0.035 | 2.6 | 0.024 | 4.4 |
| Albumin <33 g/L | 0.004 | 3.7 | 0.002 | 6.5 |
| cFLC >65 mg/L | 0.04 | 2.3 | 0.015 | 7.1 |
| Age >75 years | <0.01 | 3.8 | n/a | n/a |

To simplify the analysis due to the broad range of causes of death observed in this population, the primary causes of death listed on the death certificates were categorised according to the WHO International Statistical Classification of Diseases and Related Health Problems 10th Revision (ICD-10).

Mann-Whitney U test, Pearson Chi-squared test, Kaplan-Meier curves and Cox regression analysis were performed using SPSS (version 19; Chicago, USA). Correlation analysis was performed using GraphPad Prism (version 5). Analysis using penalised smoothing splines (P-splines) was performed to assess the association of risk of death with cFLC concentration (SAS version 9.1.3; SAS Institute, Cary, USA).

### Results

### Patient Demography

Median age of the patients was 60 years old (range 26-87), male/female ratio (216:307) (Table 1). 122 were hospitalised, 367 were outpatient referrals and 38 were primary care patients. The known malignancies recorded in this population included patients with CLL, cancers and lymphomas (FIG. 1). 11/32 patients with known malignancies including CLL, cancer and myelodysplastic syndrome died. Patients with impaired kidney function (N=128, eGFR <60 ml/min/1.73 m², equivalent to CKD stage 3 and above) had higher concentrations of cFLC vs CKD stage 1 and 2 patients (median 64.6 mg/L vs 35.5 mg/L, p<0.001).

### Early Death and Risk Factor Analysis

Over the 4.5 years of follow-up, there were 99 deaths (=18.8% mortality). A Kaplan-Meier curve revealed that almost a third of the deaths (29%) occurred within the first 100 days (FIG. 2). For this reason, subsequent analyses were performed separately for early deaths (<100 days) and all deaths (throughout follow-up).

In this study, cFLC were evaluated as a potential new ‘mortality predictor’ biomarker, in addition to established risk factors including, ESR, CRP, albumin, eGFR, and age.

TABLE 3
Patient characteristics: Continuous variables show median (95% ile range).
eGFR = estimated glomerular filtration rate (* eGFR values above
120 ml/min/1.73 m²). ALP = alkaline phosphatase.
ALT = alanine aminotransferase transaminase. WCC = white
cell count. CRP = C reactive protein. ESR = erythrocyte
sedimentation rate.

| | All patients N = 527 | |
|---|---|---|
| Age (yrs) | 59.8 | (26.06-87.18) |
| N | 527 | |
| Gender (male %) | 216 | (41.0%) |
| N | 523 | |
| Ethnicity (%) | | |
| Caucasian | 64.5 | |
| Asian | 13.7 | |
| Afro-Caribbean | 4.7 | |
| Other | 17.1 | |
| N | 527 | |
| Mortality during study period (%) | 18.8% | |
| N | 99 | |
| eGFR (mL/min/1.73 m²) | 78.7 | (12.5-139) |
| N | 494 | |
| Bilirubin (μmol/L) | 8 | (3-48.5) |
| N | 443 | |
| ALP (U/L) | 82 | (46-257.5) |
| N | 480 | |
| ALT (U/L) | 25 | (9.05-175.4) |
| N | 441 | |
| Total protein (g/L) | 74 | (57-87.9) |
| N | 443 | |
| Calcium (μmol/L) | 2.29 | (1.97-2.6) |
| N | 417 | |
| Haemoglobin (g/dL) | 13.3 | (7.95-16.6) |
| N | 509 | |
| WCC (×10³/mm³) | 7.2 | (3.3-20.88) |
| N | 509 | |
| Neutrophils (×10³/mm³) | 4.3 | (1.6-14.3) |
| N | 509 | |
| FLC κ (mg/L) | 18.2 | (8.23-102.8) |
| N | 527 | |
| FLC λ (mg/L) | 20.4 | (9-92.04) |
| N | 527 | |
| FLC κ/λ ratio | 0.9 | (0.48-1.7) |
| N | 527 | |
| cFLC (mg/L) | 39 | (18.76-205) |
| N | 527 | |
| Serum creatinine (μmol/L) | 79 | (50-415.7) |
| N | 497 | |
| CRP (mg/L) | 6 | (1-195.65) |
| N | 348 | |
| ESR (mm/hr) | 17 | (4-92.45) |
| N | 390 | |
| Albumin (g/L) | 44 | (28.88-51) |
| N | 474 | |
| IgG (g/L) | 11.8 | (5.71-23.9) |
| N | 527 | |
| IgA (g/L) | 2.57 | (0.9-7.77) |
| N | 525 | |
| IgM (g/L) | 0.94 | (0.23-3.06) |
| N | 523 | |
| Summated IgG/A/M (g/L) | 15.82 | (8.78-31.61) |

| | |
|---|---|
| N | 522 |

### Early Risk Factors: Univariate Analysis

Univariate analysis identified albumin<33 g/L, CRP>10 mg/L, ESR>12 mm/hr, eGFR<30 mL/min/1.73 m², age>75 years, elevated cFLC and gender (male) as being significant (p<0.05) predictors of mortality within 100 days (Table 2a).

The relative risk of death increased proportionally with increasing cFLC concentrations (FIG. 3); patients with a lower cFLC concentration (<50 mg/L) had a reduced risk of death compared to patients with a higher concentration (>50 mg/L). ROC analysis indicated 65 mg/L as the optimum cut-off for identifying patients with a greater risk of death.

### Early Risk Factors: Multivariate Analysis

Using multivariate analysis, only cFLC>65 mg/L, albumin concentrations<33 g/L and eGFR<30 mL/min/1.73 m² were independently associated with mortality within 100 days (Table 2b). cFLC was shown to correlate moderately with these factors, with the strongest correlation observed between cFLC and albumin (r=−0.48, p<0.001) (FIG. 4).

cFLC Risk Stratification Model

A simple risk stratification model was constructed combining albumin<33 g/L and/or cFLC>65 mg/L as risk factors. This separated patients with 0 (hazard ratio (HR)=1)), 1 (HR=4.2; CI=2.6-6.7, p<0.001) or 2 (HR=24; CI=13.2-43.8, p<0.001) risk factors, (termed the Combylite Risk Score; FIG. 5). Of the patients who died within 100 days, 86% had either 1 or both risk factors. For deaths throughout the period of follow-up, 56% of the patients had either 1 or 2 risk factors. For the same risk factors analysed independently, the proportion of associated deaths within 100 days and throughout follow-up were 50% and 24% for albumin<33 g/L and 73% and 50% for cFLC>65 mg/L.

### Late Risk Factors

For all deaths within the period of follow-up, univariate analysis identified the same risk factors as for death within 100 days, with the exception of male gender (Table 2a). The independent risk factors identified by multivariate analysis were eGFR, albumin, cFLC and age (Table 2b). Apart from age, which was not identified as an independent risk factor for death within 100 days, the variables had lower HRs and significance levels than for the prediction of early deaths.

### Causes of Death

The most frequent classifications for the primary cause of death were ‘circulatory’, ‘respiratory’, with ‘neoplasm’ and ‘digestive’ being the next common. For circulatory, respiratory and digestive deaths, the incidence (%) was significantly higher in patients with cFLC>65 mg/L (FIG. 6). The same predominant causes of death were seen for those who died in <100 or >100 days (data not presented).

Circulatory deaths comprised mostly strokes and heart attack/failure whilst infections/respiratory deaths were predominantly attributed to pneumonia. Digestive deaths included multiple organ failure, gastrointestinal haemorrhage and several forms of liver disease. Deaths due to neoplasms were not significantly associated with high cFLC.

### Discussion

Here we have demonstrated that, in a hospital referral population, elevated cFLC concentrations were associated with increased risk of mortality. This extends the preliminary reports of cFLC prognosis in general populations [Eisele, L, Durig, J, Huttman, A et al. Polyclonal free light chain elevation and mortality in the German Heinz Nixdorf Recall Study. Blood 2010; 116; 3903a-, Dispenzieri, A, Katzmann, JA, Kyle, R A et al. Use of nonclonal serum immunoglobulin free light chains to predict overall survival in the general population. Mayo Clin Proc 2012; 87; 517-523]. Furthermore, this prognostic value was independent of other previously defined biomarkers notably, decreased albumin [Corti, M C, Guralnik, J M, Salive, M E et al. Serum albumin level and physical disability as predictors of mortality in older persons. JAMA 1994; 272; 1036-1042.], elevated ESR [Danesh, J, Collins, R, Peto, R et al. Haematocrit, viscosity, erythrocyte sedimentation rate: meta-analyses of prospective studies of coronary heart disease. Eur Heart J 2000; 21; 515-520.], reduced eGFR [Go, A S, Chertow, GM, Fan, D et al. Chronic kidney disease and the risks of death, cardiovascular events, and hospitalization. N Engl J Med 2004; 351; 1296-1305.] and elevated CRP [Koenig, W, Khuseyinova, N, Baumert, J et al. Prospective study of high-sensitivity C-reactive protein as a determinant of mortality: results from the MONICA/KORA Augsburg Cohort Study, 1984-1998. Clin Chem 2008; 54; 335-342.].

Patients from a diverse background were included in this study, including patients from primary care, out-patients and hospitalised groups. The purpose of selecting such a cohort was to avoid selection bias and as a pilot study indicator for a larger prospective study. In this population, there was an increased frequency of death within the first 100 days and cFLC had the largest HR associated with outcome within this period (HR=7.1), with 73% of deceased patients having a cFLC>65 mg/L. Cardiovascular disease (CVD) accounted for a large proportion of these deaths (12/29, 41%) but CRP was not an independent risk factor for mortality. A simple, 3-tiered, risk-stratification model incorporating reduced serum albumin and/or elevated cFLC identified 86% of all cause mortality within 100 days, suggesting this could constitute a sensitive and very effective method of identifying patients with high risk of early death who might benefit from prompt and more detailed further investigation. While this risk stratification has been demonstrated with a patient population who all had SPE requests, a more appropriate application might be with patients referred to a medical assessment unit.

The prognostic value of cFLC diminished during the course of follow up (from HR=7.1, p=0.015 at 100 days to HR=2.3, p=0.04 after 4.5 years) and indeed predicting overall outcome over such a long period of time may not suggest many practical applications. However, in 25% of these patients, death was attributed to CVD related causes. It could, therefore, be argued that it would be appropriate to evaluate cFLC as a cardiovascular risk factor alongside other established evaluations such as blood pressure and lipoprotein concentrations.

We surmise that it is the combination of pathological influences on FLC production and/or the different routes of FLC clearance that result in the association of raised cFLC with increased all-cause mortality rates. A simplified mechanistic model for elevated serum free light chains is shown in FIG. 7. Whilst this may partially describe the factors which may influence FLC concentrations it does not reflect the complexity of the system and further studies are required (FIG. 7). It has already been noted that increased polyclonal FLC production is associated with disease activity/outcomes in autoimmune diseases, infections, aging and chronic kidney disease; although it is noteworthy in this data cFLC is independent of both age and renal function [Gottenberg, J E, Aucouturier, F, Goetz, J et al. Serum immunoglobulin free light chain assessment in rheumatoid arthritis and primary Sjogren's syndrome. Ann Rheum Dis 2007; 66; 23-27, Hoffman, U, Opperman, M, Kuchler, S et al. Free immunoglobulin light chains in patients with rheumatic diseases. Z Rheumatol 2003; 62;Fr40a-, Aggarwal, R, Sequeira, W, Kokebie, R et al. Serum free light chains as biomarkers for systemic lupus erythematosus disease activity. Arthritis Care Res 2011; 63; 891-898, Hutchison, C A, Harding, S, Hewins, P et al. Quantitative assessment of serum and urinary polyclonal free light chains in patients with chronic kidney disease. Clin J Am Soc Nephrol 2008; 3; 1684-1690.]. Increased polyclonal production, suggesting general B-cell stimulation, is also associated with some haematological malignancies and has been reported to be prognostic for Hodgkin's disease [De Filippi, R, Russo, F, Iaccarino, G et al. Abnormally elevated levels of serum free-immunoglobulin light chains are frequently found in classic Hodgkin Lymphoma (cHL) and predict outcome of patients with early stage disease. Blood 2009; 114; 267a-], non-Hodgkin's lymphoma [Landgren, O, Goedert, J J, Rabkin, C S et al. Circulating serum free light chains as predictive markers of AIDS-related lymphoma. J Clin Oncol 2010; 28; 773-779., Maurer, M J, Micallef, I N, Cerhan, J R et al. Elevated serum free light chains are associated with event-free and overall survival in two independent cohorts of patients with diffuse large B-cell lymphoma. J Clin Oncol 2011; 29; 1620-1626.] and chronic lymphocytic leukaemia [Morabito, F, De Filippi, R, Laurenti, L et al. The total amount of kappa plus lambda serum immunoglobulin free light chains (sFLC κ+λ) is a powerful independent predictor of time to first treatment in chronic lymphocytic leukemia (CLL) and allows definition of a novel prognostic scoring system: a study of 449 therapy-naive patients. Blood 2010; 116; 2437a-, Pratt, G, Harding, S, Fegan, C et al. Serum FLC levels at presentation have independent prognostic significance in CLL and levels above 50 mg/L identify patients with progressive disease. Blood 2009; 114; 2355a-, Maurer, MJ, Cerhan, JR, Katzmann, J A et al. Monoclonal and polyclonal serum free light chains and clinical outcome in chronic lymphocytic leukemia. Blood 2011; 118; 2821-2826.]. However, the most common cause for increased polyclonal FLC is probably reduced clearance due to renal impairment [Hutchison, C A, Harding, S, Hewins, P et al. Quantitative assessment of serum and urinary polyclonal free light chains in patients with chronic kidney disease. Clin J Am Soc Nephrol 2008; 3; 1684-1690.] and CKD is well known to be associated with increased morbidity and mortality, particularly from CVD [Go, A S, Chertow, G M, Fan, D et al. Chronic kidney disease and the risks of death, cardiovascular events, and hospitalization. N Engl J Med 23-9-2004; 351; 1296-1305.]. FLC are also cleared via pinocytosis by cells of the reticulo-endothelial system. The liver is a major site for this removal and it is possible that reduced clearance via this route contributes to the increased FLC concentrations seen in some liver disease patients [Assi, LK, Hughes, RG, Gunson, B et al. Abnormally elevated serum free light chains in patients with liver disease. Journal of Hepatology 2010; 51; 5440-5441-]. While renal clearance of FLC is the dominant mechanism in healthy subjects, a reduction in reticulo-endothelial clearance is likely to have a significant influence on cFLC concentrations if there is already a reduction in eGFR.

Monoclonal FLC measurements are associated with adverse outcome in the majority of monoclonal gammopathies studied. Polyclonal FLC levels have been associated with adverse outcome in other haematological malignancies and as markers of malignant transformation. Our study highlights a potential utility for these enigmatic molecules in all cause mortality, both in early detection of adverse outcome (<100 days) and over a 4.5 year follow-up.

### MEWS Assessment

### Study Design

This study is a prospective study designed to investigate the role of cFLCs in a cohort of patients attending the Medical Admissions Unit (MAU) at New Cross Hospital, Wolverhampton. Patients attending MAU, who will be eligible for MEWS/EWS testing (including assessment of body temperature, heart rate, urine testing and tests for consciousness). As part of their initial assessment, Combylite will also be used to determine their cFLC levels. As part of the patient's routine assessment in MAU, patients will be providing blood samples, together with other tests including X-rays and scans. Once the sample has been sent to Haematology, it will be used to determine cFLC concentrations using the Combylite test for cFLC from The Binding Site Group Limited. Variables which constitute MEWS/EWS will also be tested, alongside any other tests which are required, depending on the clinical complications the patient is presenting. Once these assessments are completed, the clinician or consultant treating the patient will decide whether the patient is fit enough to be sent home. Alternatively, the patient may be admitted into hospital for further treatment. This may include admittance onto an emergency ward (such as a high dependency unit or intensive care unit), admittance onto a general ward, or the patient may require surgery. Any abnormal results will be followed up as they would normally have been, by the requesting clinician. Once testing is complete, follow up assessments of the patient will be made after 3 months, 6 months and 1 year. The primary outcome to be assessed in this study will be whether the patient has died or is alive at the following time points: 3 months, 6 months and 1 year. These outcomes will then be compared to the patient's MEWS/EWS scores (which will be determined at presentation, discharge from hospital and the worst score) and cFLCs result, to determine whether MEWS/EWS together with cFLCs assessment would have been beneficial in determining how the patient should be treated vs the MEWS/EWS score alone.

All patients attending the MAU at New Cross Hospital, Wolverhampton will be eligible for inclusion into the study. A maximum of 3000 patients will be recruited into the study, and this has been calculated by taking into account the following:
1) Firstly, there will be an average of 30 patients assessed in MAU each day and the pilot study will run for 3 months, averaging ˜3000 patients. This is based on the expected number of patients to be seen within that time period.
2) Secondly, a power calculation has been used to determine the number of patients required based on results obtained from a previous study (6). In brief, this study included a hospital referral population who were referred to the haematology department. A serum protein electrophoresis (SPE) test had been requested, between Nov. 8, 2005 and Jan. 10, 2006 (N=723 serum samples). Samples from paediatric patients, patients on immunoglobulin replacement, second and subsequent samples from the same patient, were excluded from the analysis. Also excluded were all patients with evidence of a monoclonal gammopathy as indicated by an abnormal FLC ratio (<0.26 or >1.65) (9) or an abnormal SPE result (if confirmed by immunofixation). This left 528 patients in the final data analysis. However, this is based solely on patients where an SPE has been requested. As an SPE will be requested in approximately 20% of patients attending MAU, to consider all patients attending MAU, the total number of patients required for this study will be approximately 3000.

There are certain patient groups who are known to express elevated FLCs, including patients with chronic kidney disease, and systemic lupus erythematosus. However, as survival (i.e. the status of whether the patient is alive or dead) will be the primary outcome analysed at each time point, these patients will not be excluded based on any currently known diagnosis. The patients cFLC result will be compared to the MEWS or EWS score to determine whether inclusion of Combylite would have had any impact on the patients outcome compared to simply using the MEWS or EWS score alone.

It is expected that, in the light of the evidence found for cFLC survival above, that the combination of MEWS and cFLC will improve the determination of the severity of symptoms in patients.

What is claimed is:

1. A method of assessing the severity of symptoms in a patient comprising:
(i) producing a triage score for the patient,
wherein the triage score is selected from the group consisting of an early warning score (EWS), a modified early warning score (MEWS), a pediatric early warning score (PEWS), a NHS early warning score (NEWS), a simple clinical score (SCS), or a rapid emergency score (REMS),
(ii) measuring an amount of free light chains (FLC), in a sample from the patient, and
(iii) assessing the severity of symptoms in the patient based upon the triage score and the amount of FLC.

2. The method according to claim 1, wherein producing the triage score comprises:
measuring one or more clinical factors of the patient selected from the group consisting of a systolic blood pressure, a heart rate, a respiratory rate, and a body temperature,
comparing each measurement to a predetermined normal value, and
producing a triage score for each measurement.

3. The method according to claim 1, wherein producing the triage score comprises:
scoring a level of one or more clinical factors in the patient selected from the group consisting of consciousness, blood oxygen level, urine output, and pain.

4. The method according to claim 1, wherein the patient has been admitted to a medical admissions unit.

5. The method according to claim 13, wherein a measured amount of cFLC above 50 mg/L indicates an increased likelihood of death of the patient within 100 days without further treatment.

6. The method according to claim 1, further comprising measuring the amount of serum albumin in the patient.

7. The method according to claim 6, wherein an amount of serum albumin of below 33 g/L indicates an increased risk of death of the patient within 100 days without further treatment.

8. The method according to claim 1, wherein the amount of FLC is determined by immunoassay using anti-free light chain antibodies or fragments thereof.

9. (canceled)

10. An assay kit comprising anti-FLC antibodies or fragments thereof, in combination with anti-albumin antibodies or fragments thereof, for use in a method according to claim 11.

11. The method according to claim 6, wherein the amount of albumin is determined by immunoassay using anti-free light chain antibodies.

12. The method according to claim 1, wherein the free light chains that are measured are combined free light chains (cFLC).

13. The method according to claim 1, further comprising diagnosing the patient.

14. The method according to claim 1, further comprising treating the patient.

15. The method according to claim 1, further comprising keeping the patient under medical observation.

16. The method according to claim 1, further comprising discharging the patient from medical supervision.

17. The method according to claim 1, wherein the sample from the patient is selected from the group consisting of whole blood, plasma, urine, serum, tissue, or other human fluids.

18. The method according to claim 1, further comprising measuring the amount of one or more markers in the patient selected from the group consisting of C-reactive protein (CRP), estimated glomerular filtration rate (eGFR), and erythrocyte sedimentation rate (ESR).

19. The method according to claim 8, wherein the antibody fragments comprise (Fab)₂ antibodies or Fab antibodies.

20. The method according to claim 8, wherein the antibodies or fragments thereof are labelled.

21. The method according to claim 8, wherein the antibodies or fragments thereof are unlabelled.
